(19) United States
(12) Patent Application Publication
(10) Pub. No.: US 2013/0158466 A1
(43) Pub. Date: Jun. 20, 2013

(54) PLASMA BRUSHES

(71) Applicants: Yark Yeon KIM, Daejeon (KR); Han Young Yu, Daejeon (KR); Yong Ju Yun, Daejeon (KR); Won Ick Jang, Daejeon (KR); Eun-ju Jeong, Daejeon (KR); Changho Han, Daegu (KR)

(72) Inventors: Yark Yeon KIM, Daejeon (KR); Han Young Yu, Daejeon (KR); Yong Ju Yun, Daejeon (KR); Won Ick Jang, Daejeon (KR); Eun-ju Jeong, Daejeon (KR); Changho Han, Daegu (KR)

(73) Assignee: Electronics and Telecommunications Research Institute, Daejeon (KR)

(21) Appl. No.: 13/619,990

(22) Filed: Sep. 14, 2012

(30) Foreign Application Priority Data
Dec. 16, 2011 (KR) 10-2011-0136594

(51) Int. Cl. A61M 37/00

(52) U.S. Cl. 604/23

(57) ABSTRACT

Plasma brushes are provided. The plasma brush includes a nozzle, a connector connected to a first end of the nozzle, a power electrode disposed at a portion of the nozzle, and a ground electrode disposed at a second end of the nozzle opposite to the connector.

### CROSS-REFERENCE TO RELATED APPLICATIONS

This U.S. non-provisional patent application claims priority under 35 U.S.C. §119 to Korean Patent Application No. 10-2011-0136594, filed on Dec. 16, 2011, the disclosure of which is hereby incorporated by reference in its entirety.

### BACKGROUND

1. Technical Field

The present disclosure herein relates to medical instruments and, more particularly, to plasma brushes used in medical instruments.

2. Description of Related Art

Biological effects of atmospheric cold plasma have been reported since the beginning of the year 2000, and many concerns are focused on the atmospheric cold plasma. It is well known that the atmospheric cold plasma can sterilize and/or disinfect microbes. Accordingly, the atmospheric cold plasma has been widely used in various areas such as air cleaners or noxious gas filters. Recently, many research findings about interactions of bio cells with plasma have been increasingly reported to accelerate or predict vitalization of new medical industry.

Stability of the plasma temperature as well as diverse structures of medical treatment regions (e.g., affected parts) may be fundamentally required to utilize atmospheric cold plasma systems as the medical instruments. In the case of plasma jet, the plasma generation devices may be more readily fabricated, but not appropriate for medical treatment of the affected parts having a large area. Thus, novel medical atmospheric cold plasma systems may be required to cure and/or treat various and wide affected parts.

### SUMMARY

Exemplary embodiments are directed to plasma brushes used in medical plasma systems.

According to some embodiments, a plasma brush includes a nozzle, a connector connected to a first end of the nozzle, a power electrode disposed at a portion of the nozzle, and a ground electrode disposed at a second end of the nozzle opposite to the connector.

In some embodiments, the nozzle may include a discharge port having a rectangular shape or an oval shape, and the rectangular shape or the oval shape may have a lateral width and a vertical height which is less than the lateral width.

In some embodiments, the power electrode may include a first metal ring surrounding an outer surface of the nozzle, and the ground electrode may include a second metal ring surrounding an outer surface of the nozzle.

In some embodiments, the first metal ring and the second metal ring may have the same vertical height.

In some embodiments, a lateral width of the first metal ring may be less than that of the second metal ring.

In some embodiments, the nozzle may include a first groove in which the first metal ring is disposed and a second groove in which the second metal ring is disposed.

In some embodiments, the nozzle may further include a protruding sill that is disposed between the first and second grooves to separate the first metal ring from the second metal ring.

In some embodiments, the plasma brush may further include a filler nozzle inserted into the discharge port. The filler nozzle may have a plurality of pores for ejecting a gas.

In some embodiments, the ground electrode may fill a space between the pores to have a grid shape or a mesh shape.

In some embodiments, the ground electrode may be disposed on a front surface of the filler nozzle, and the plasma brush may further include a first coating layer covering a front surface of the ground electrode opposite to filler nozzle.

In some embodiments, the power electrode may include a plurality of needles disposed in the discharge port.

In some embodiments, the plasma brush may further include a conductive adhesive agent that electrically connects the power needles to each other.

In some embodiments, the plasma brush may further include a second coating layer that protects the power needles.

In some embodiments, the nozzle may include a dielectric material.

In some embodiments, the dielectric material may include an alumina material or a ceramic material.

In some embodiments, the connector may include a tube having a plurality of channel holes through which a gas flows.

In some embodiments, the tube may include an insulation rod surrounded by the plurality of channel holes.

In some embodiments, the connector may further include a third coating layer protecting the tube.

### BRIEF DESCRIPTION OF THE DRAWINGS

Embodiments of the inventive concept will become more apparent in view of the attached drawings and accompanying detailed description.

FIG. 1 is a plan view illustrating a plasma brush according to an exemplary embodiment.

FIGS. 2 and 3 are a side view and a front view of the plasma brush illustrated in FIG. 1, respectively.

FIG. 4 is a plan view illustrating a plasma brush according to another exemplary embodiment.

FIG. 5 is an enlarged plan view illustrating a filler nozzle and a ground electrode of FIG. 4.

FIG. 6 is a side view of the plasma brush illustrated in FIG. 4.

FIG. 7 is an enlarged side view illustrating a power electrode and a filler nozzle of FIG. 6.

FIG. 8 is a front view illustrating a nozzle and a filler nozzle of FIG. 4.

FIG. 9 is a plan view illustrating a plasma brush according to yet another exemplary embodiment.

FIG. 10 is an enlarged plan view illustrating power needles and a ground electrode of FIG. 9.

FIG. 11 is a side view of the plasma brush illustrated in FIG. 9.

FIG. 12 is an enlarged side view illustrating a nozzle and a filler nozzle of FIG. 10.

FIG. 13 is a side cross sectional view illustrating a gas supply conduit and a nozzle connector of a plasma brush according to still another exemplary embodiment.

FIGS. 14 and 15 are cross sectional views of the gas supply conduit and the nozzle connector illustrated in FIG. 13, respectively.

### DETAILED DESCRIPTION OF THE EMBODIMENTS

The inventive concept will now be described more fully hereinafter with reference to the accompanying drawings, in which exemplary embodiments of the inventive concept are shown. The advantages and features of the inventive concept and methods of achieving them will be apparent from the following exemplary embodiments that will be described in more detail with reference to the accompanying drawings. It should be noted, however, that the inventive concept is not limited to the following exemplary embodiments, and may be implemented in various forms. Accordingly, the exemplary embodiments are provided only to disclose the inventive concept and let those skilled in the art know the category of the inventive concept. The same reference numerals or the same reference designators denote the same elements throughout the specification. In the drawings, the exemplary embodiments of the inventive concept are not limited to the specific examples provided herein and the thicknesses of layers and regions are exaggerated for clarity.

The terminology used herein is for the purpose of describing particular embodiments only and is not intended to limit the invention. As used herein, the singular terms “a,” “an” and “the” are intended to include the plural forms as well, unless the context clearly indicates otherwise. As used herein, the term “and/or” includes any and all combinations of one or more of the associated listed items. It will be understood that the terms “has”, “having”, “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof. It will be further understood that when an element such as a layer, region or substrate is referred to as being “on” another element, it can be directly on the other element or intervening elements may be present. In contrast, the term “directly” means that there are no intervening elements. Similarly, it will be also understood that when an element is referred to as being “connected” or “coupled” to another element, it may be directly connected or coupled to the other element or intervening elements may be present.

Additionally, the embodiment in the detailed description will be described with plan views and/or sectional views as ideal exemplary views of the inventive concept. Accordingly, shapes of the exemplary views may be modified according to manufacturing techniques and/or allowable errors. Therefore, the embodiments of the inventive concept are not limited to the specific shape illustrated in the exemplary views, but may include other shapes that may be created according to manufacturing processes. For example, a region illustrated as a rectangle may have rounded or curved features. Thus, areas exemplified in the drawings have general properties, and are used to illustrate specific shapes of elements. Accordingly, this should not be construed as limited to the scope of the inventive concept.

It will be also understood that although the terms first, second, third etc. may be used herein to describe various elements, these elements should not be limited by these terms. These terms are only used to distinguish one element from another element. Thus, a first element in some embodiments could be termed a second element in other embodiments without departing from the teachings of the inventive concepts. Exemplary embodiments of aspects of the present inventive concept explained and illustrated herein include their complementary counterparts.

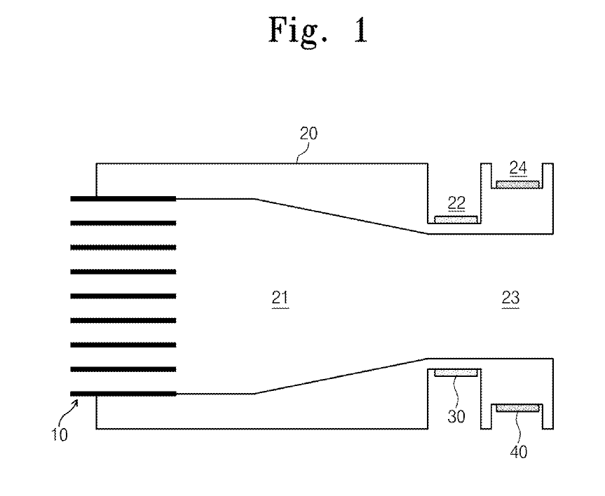

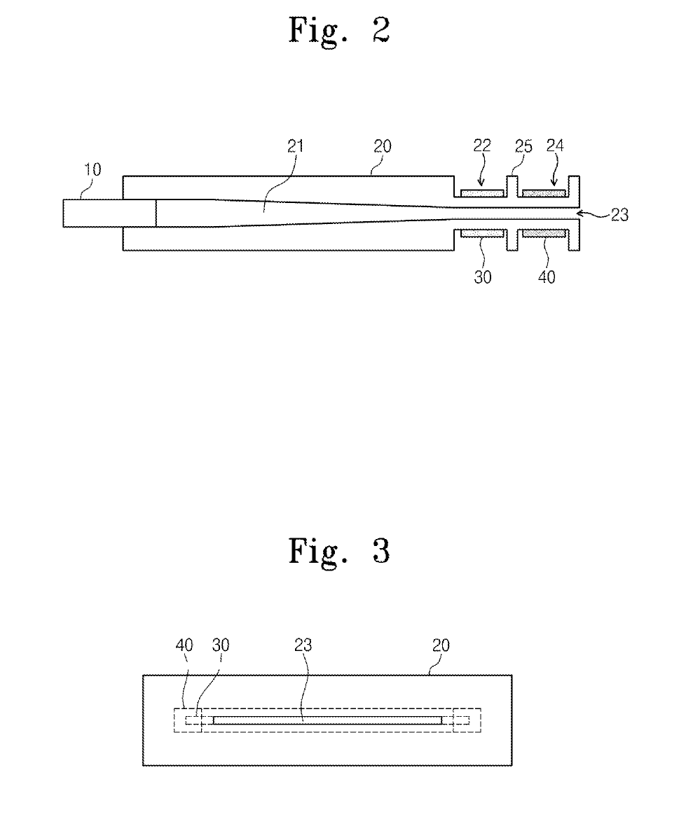

FIG. 1 is a plan view illustrating a plasma brush according to an exemplary embodiment, and FIGS. 2 and 3 are a side view and a front view of the plasma brush illustrated in FIG. 1, respectively.

Referring to FIGS. 1, 2 and 3, a plasma brush according to an exemplary embodiment may include a nozzle 20 having a discharge port 23 for ejecting a plasma gas. The discharge port 23 may have a rectangular shape or an oval shape in a front view. The rectangular shape or the oval shape may have a vertical height and a lateral width which is greater than the vertical height when viewed from a front view. The plasma gas may be generated by high frequency electrical power applied between a power electrode 30 and a ground electrode 40 which are disposed on an outer surface of the nozzle 20.

The plasma brush according to the present exemplary embodiment may provide a large area corresponding to the cross sectional area of the discharge port 23 having the rectangular shape or the oval shape with the plasma gas.

The nozzle 20 may be formed of a dielectric material, for example, an alumina material or a ceramic material. The nozzle 20 may have an inlet 21 extending from a nozzle connector 10. The nozzle 20 may convert a pressure energy of a gas into a velocity energy thereof, thereby ejecting the gas at a high speed. A cross sectional area of the discharge port 23 may be less than that of the inlet 21. The gas may include, for example, a helium gas, an argon gas, a nitrogen gas or an oxygen gas. In an exemplary embodiment, the discharge port 23 may have a rectangular cross sectional opening, as illustrated in FIG. 3.

A portion of the nozzle 20 may be inserted into the power electrode 30 as well as the ground electrode 40. For example, the power electrode 30 may include a first ring (not shown) that surrounds a portion of the outer surface of the nozzle 20 and the ground electrode 40 may include a second ring (not shown) that surrounds another portion of the outer surface of the nozzle 20. A high frequency electric power signal outputted from a power supply (not shown) may be applied to the power electrode 30, and the ground electrode 40 may be connected to a ground terminal. Thus, the gas introduced into the nozzle 20 may be excited into a plasma state by the high frequency electric power. Here, the gas excited into a plasma state may be referred to as a plasma gas or a plasma fume. Although not shown in the drawings, the power electrode 30 may be electrically connected to the power supply through a current stabilization resistor. The current stabilization resistor may prevent an arc discharge phenomenon which is due to the high frequency power.

The nozzle 20 may include a first groove 22 for fixing the power electrode 30 and a second groove 24 for fixing the ground electrode 40. A protruding sill 25 may be disposed between the first groove 22 and the second groove 24. The protruding sill 25 may electrically insulate the power electrode 30 from the ground electrode 40. A distance round the first groove 22 may be less than a distance round the second groove 24.

Referring again to FIG. 1, the first groove 22 and/or the second groove 24 may have a rectangular shape or an oval shape in a front view. In an exemplary embodiment, a width of the first groove 22 may be less than that of the second grove 24, as illustrated in FIG. 1. That is, a width of the first ring may be different from that of the second ring. A width of the power electrode 30 composed of the first ring may also be less than that of the ground electrode 40 composed of the second ring. The nozzle 20 may eject a plasma gas that is generated by the high frequency electric power applied between the power electrode 30 and the ground electrode 40.

Referring again to FIG. 2, the first and second grooves 22 and 24 may have the same vertical height. That is, the power electrode 30 and the ground electrode 40 may have the same vertical height. In an exemplary embodiment, the discharge port 23 of the nozzle 20 may have a rectangular and flat opening which is laterally wide in a front view. Thus, the plasma gas may be ejected onto a portion having a large area through the rectangular discharge port 23.

In conclusion, the plasma brushes according to the above exemplary embodiments may provide a large areal plasma gas.

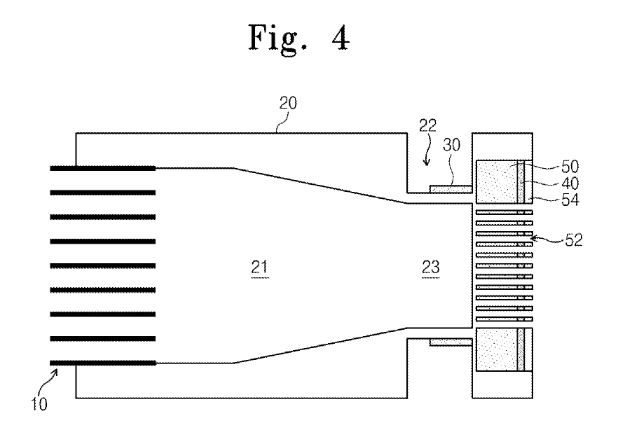

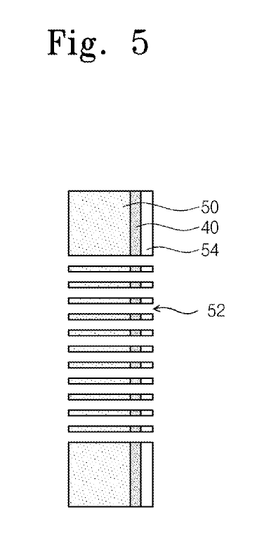

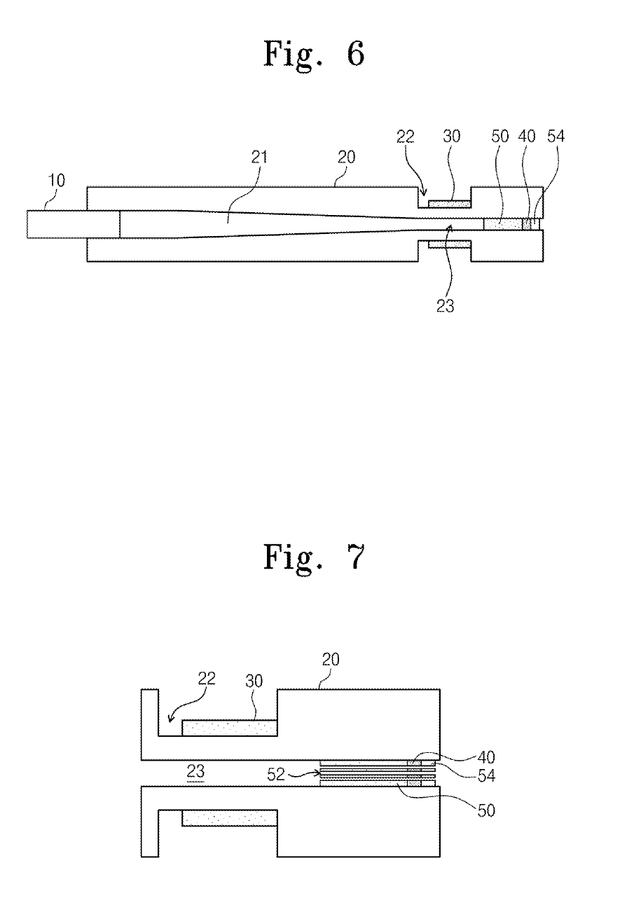

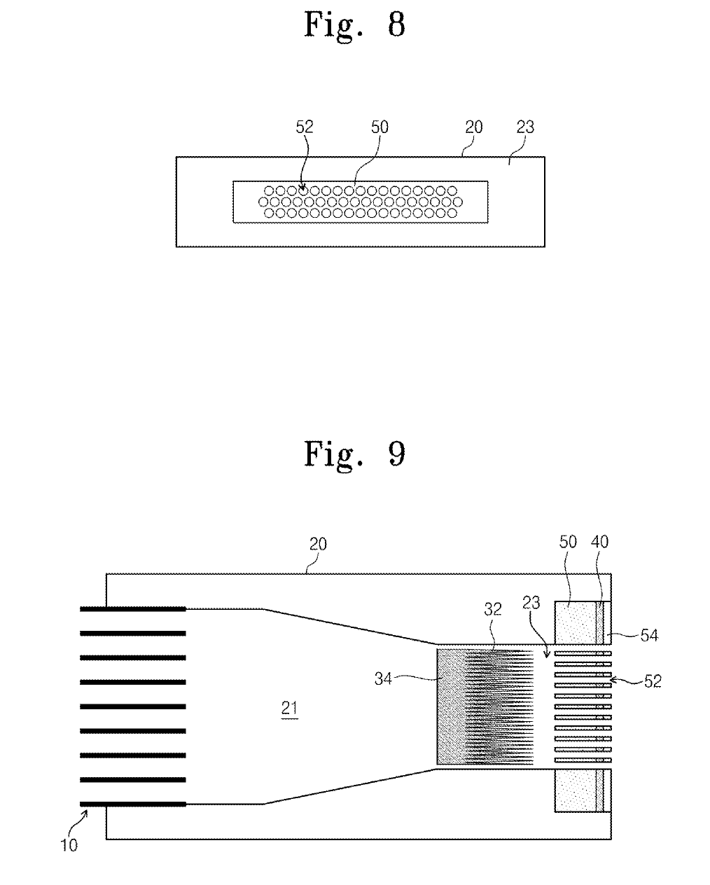

FIG. 4 is a plan view illustrating a plasma brush according to another exemplary embodiment. FIG. 5 is an enlarged plan view illustrating a filler nozzle and a ground electrode of FIG. 4, and FIG. 6 is a side view of the plasma brush illustrated in FIG. 4. In addition, FIG. 7 is an enlarged side view illustrating a power electrode and a filler nozzle of FIG. 6, and FIG. 8 is a front view illustrating a nozzle and a filler nozzle of FIG. 4.

Referring to FIGS. 4, 5, 6, 7 and 8, a plasma brush according to the present exemplary embodiment may include a filler nozzle 50 having a plurality of pores 52 therein. The filler nozzle 50 may be inserted into the discharge port 23. The ground electrode 40 may be disposed between the pores 52 of the filler nozzle 50. A plasma gas generated by high frequency electric power applied between the power electrode 30 and the ground electrode 40 may be ejected through the pores 52 of the filler nozzle 50.

Thus, the plasma brush according to the present exemplary embodiment may provide a large areal plasma gas.

The pores 52 of the filler nozzle 50 may correspond to the discharge port 23 disposed in an end of the nozzle 20. Thus, the pores 52 may be arrayed in a rectangular area or an oval area according to the shape of the discharge port 23. The pores 52 may be uniformly arrayed such that distances between the adjacent pores 52 are equal to each other.

The ground electrode 40 may fill a space between the pores 52, thereby having a grid shape or a mesh shape in a front view. The ground electrode 40 may have a rectangular shape or an oval shape in a front view and may have a lateral width which is greater than a lateral width of the power electrode 30 (see FIGS. 4 and 5). In contrast, the ground electrode 40 may have a vertical height which is less than a vertical height of the power electrode 30 (see FIGS. 6 and 7).

A front surface of the filler nozzle 50 may be covered with the ground electrode 40, and a front surface of the ground electrode 40 may be covered with a first coating layer 54. The first coating layer 54 may include a dielectric layer such as an aluminum oxide (e.g., alumina) layer, a silicon oxide layer or a silicon nitride layer. Alternatively, the first coating layer 54 may include a plastic material or a polymer material that has an excellent insulating property.

The power electrode 30 may include a first ring having a rectangular shape or an oval shape, which surrounds a first groove 22 of the nozzle 20. A gas passing through the nozzle 20 may be excited into a plasma state by the high frequency electric power applied between the power electrode 30 and the ground electrode 40. The plasma gas may be ejected onto a large area, which is widely spread to have a rectangular shape or an oval shape, through the pores 52 of the filler nozzle 50.

Accordingly, the plasma brush according to the present exemplary embodiment may provide a large areal plasma gas.

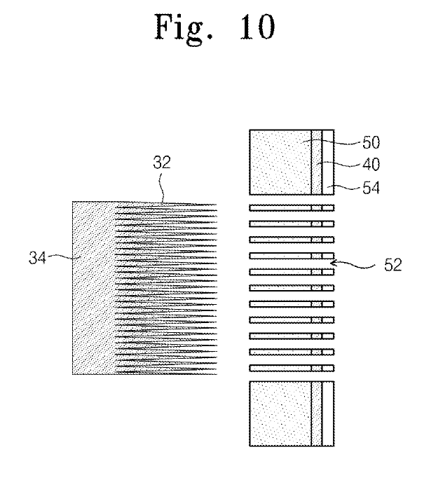

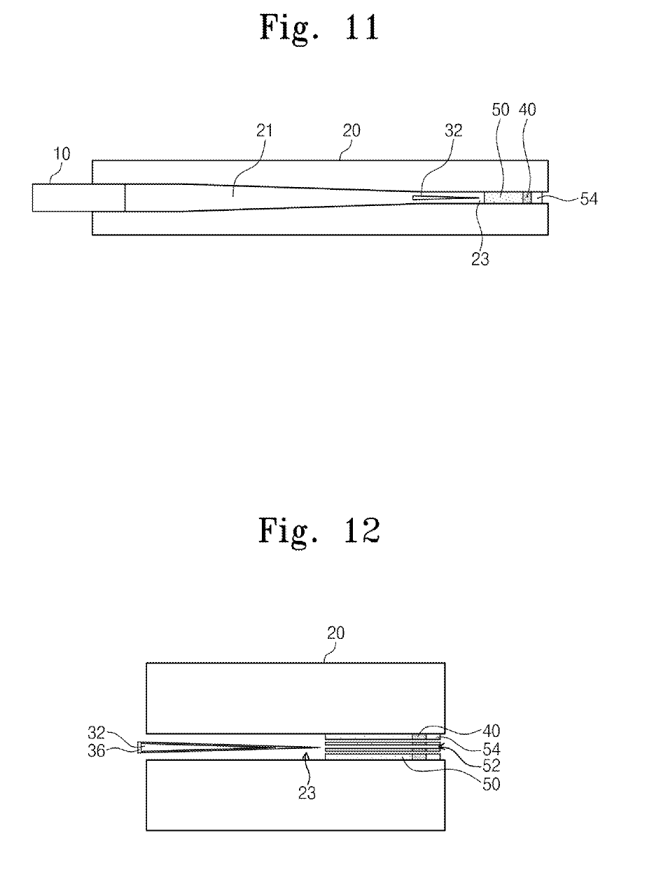

FIG. 9 is a plan view illustrating a plasma brush according to yet another exemplary embodiment. FIG. 10 is an enlarged plan view illustrating power needles and a ground electrode of FIG. 9, and FIG. 11 is a side view of the plasma brush illustrated in FIG. 9. Further, FIG. 12 is an enlarged side view illustrating a nozzle and a filler nozzle of FIG. 10.

Referring to FIGS. 8, 9, 10 and 11, a plasma brush according to the present exemplary embodiment may include power needles 32 disposed in the discharge port 23 of the nozzle 20. The plasma brush according to the present exemplary embodiment may also include the filler nozzle 50 having the plurality of pores 52 therein, as described in the exemplary embodiment of FIGS. 4 to 8. The pores 52 may be arrayed in a rectangular area or an oval area according to the shape of the discharge port 23. The power needles 32 may be disposed in the same direction as the pores 52 of the filler nozzle 50 disposed in the discharge port 23.

Accordingly, the plasma brush according to the present exemplary embodiment may also provide a large areal plasma gas. The power needles 32 may correspond to the power electrodes 30 of the previous exemplary embodiments.

The power needles 32 may be disposed in the nozzle 20. The power needles 32 may be exposed to the plasma gas that flows through the nozzle 20. The power needles 32 may be electrically connected to each other by a conductive adhesive agent and may be covered with a second coating layer 34. Thus, the second coating layer 34 may protect the power needles 32 from the plasma gas. The power needles 32 may also generate a plasma gas using a high frequency electric power. The high frequency electric power used in the present exemplary embodiment may be lower than the high frequency electric power used in the previous exemplary embodiments.

Referring to FIGS. 10, 11 and 12, the ground electrode 40 may be configured to have a grid shape or a mesh shape that fills a space between the pores 52 of the filler nozzle 50. A front surface of the filler nozzle 50 may be covered with the ground electrode 40, and a front surface of the ground electrode 40 may be covered with the first coating layer 54. The first coating layer 54 may include a dielectric layer such as an aluminum oxide layer, a silicon oxide layer or a silicon nitride layer. Alternatively, the first coating layer 54 may include a plastic material or a polymer material that has an excellent insulating property. A gas passing through the discharge port 23 of the nozzle 20 may be excited into a plasma state by the high frequency electric power applied between the power needles 32 and the ground electrode 40. The plasma gas may be ejected onto a large area, which is widely spread to have a rectangular shape or an oval shape, through the pores 52 of the filler nozzle 50.

Accordingly, the plasma brush according to the present exemplary embodiment may also provide a large areal plasma gas.

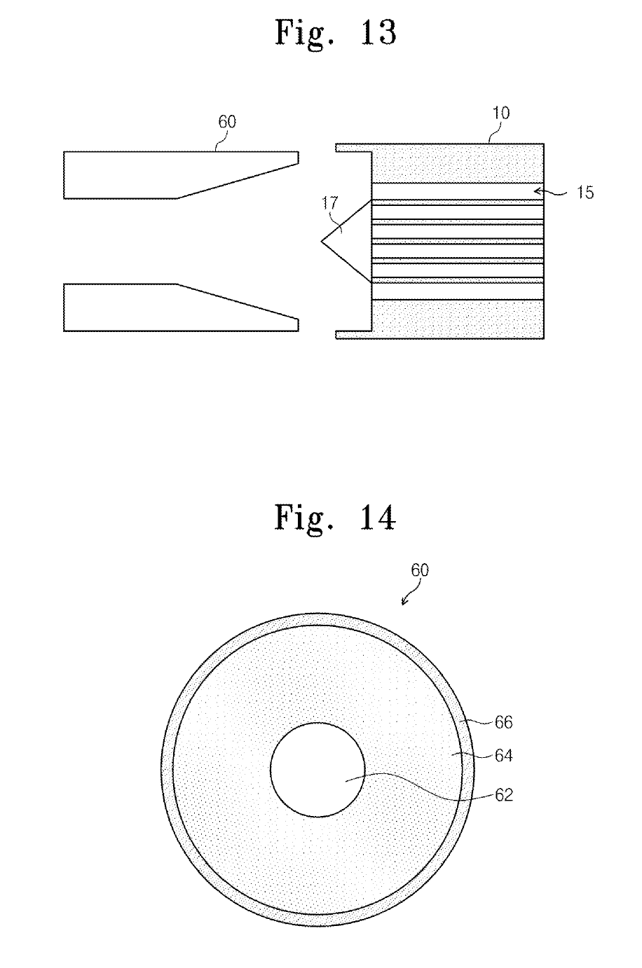

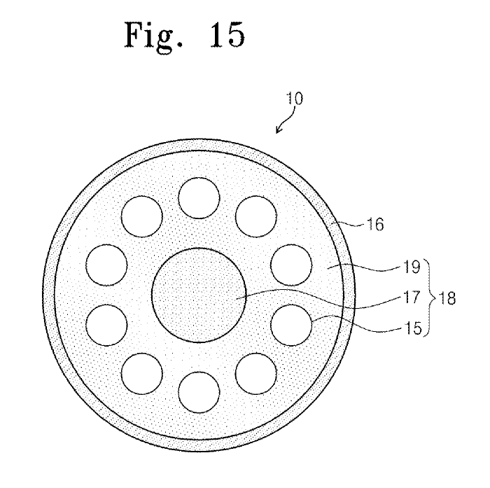

FIG. 13 is a side cross sectional view illustrating a gas supply conduit and a nozzle connector of a plasma brush according to still another exemplary embodiment. FIGS. 14 and 15 are cross sectional views of the gas supply conduit and the nozzle connector illustrated in FIG. 13, respectively.

Referring to FIGS. 1, 13, 14 and 15, a gas supply conduit 60 may include a first tube 64 having a hollow hole 62 through which a gas flows and a fourth coating layer 66 surrounding an outer surface of the first tube 64. An end of the first tube 64 may extend to have a funnel-shaped hole therein. A vertical cross sectional area of the funnel-shaped hole may be gradually increased toward the end of the first tube 64. The gas supply conduit 60 may be connected to a nozzle connector 10.

The nozzle connector 10 may be inserted into the inlet 21 of the nozzle 20. The nozzle connector 10 may include a second tube 18 and a third coating layer 16 coated on an outer surface of the second tube 18, and the second tube 18 may have an insulation rod 17 and a plurality of channel holes 15 disposed around the insulation rod 17. The channel holes 15 may be disposed to be symmetrical with respect to the insulation rod 17 when viewed from a cross sectional view. In an exemplary embodiment, the number of the channel holes 15 may be ten, as illustrated in FIG. 15. An end of the first tube 64 may be inserted into the second tube 18. The insulation rod 17 of the second tube 18 may be inserted into the hollow hole 62. The third coating layer 16 may be formed on the outer surface of the second tube 18, as described above.

Each of the first and second tubes 64 and 18 may include a dielectric material, a plastic material, a rubber material or a polymer material. The plasma brush according to the present exemplary embodiment may introduce a gas having a uniform pressure into the inlet 21 because of the presence of the gas supply conduit 60 and the nozzle connector 10. Although not shown in the drawings, a plurality of valves may be installed in respective ones of the channel holes 15 to independently control gas flow rates in the channel holes 15.

Accordingly, the gas supply conduit 60 and the nozzle connector 10 may produce a gas having a uniform pressure and may supply the uniform gas to the nozzle 20.

According to the exemplary embodiments set forth above, a nozzle may include a discharge port having a rectangular shape or an oval shape, and the rectangular shape or the oval shape may have a vertical height and a lateral width which is greater than the vertical height when viewed from a front view. Further, a filler nozzle may be disposed in the discharge port, and the filler nozzle may include a plurality of pores therein. The pores may be arrayed in a rectangular area or an oval area according to the shape of the discharge port.

Therefore, a plasma brush according to the exemplary embodiments may provide a large areal plasma gas or a large areal plasma fume.

While the inventive concept has been described with reference to example embodiments, it will be apparent to those skilled in the art that various changes and modifications may be made without departing from the spirit and scope of the inventive concept. Therefore, it should be understood that the above embodiments are not limiting, but illustrative. Thus, the scope of the inventive concept is to be determined by the broadest permissible interpretation of the following claims and their equivalents, and shall not be restricted or limited by the foregoing description.

What is claimed is:

1. A plasma brush comprising:
a nozzle;
a connector connected to a first end of the nozzle;
a power electrode disposed at a portion of the nozzle; and
a ground electrode disposed at a second end of the nozzle opposite to the connector.

2. The plasma brush of claim 1, wherein the nozzle includes a discharge port having a rectangular shape or an oval shape, and the rectangular shape or the oval shape has a lateral width and a vertical height which is less than the lateral width.

3. The plasma brush of claim 2, wherein the power electrode includes a first metal ring surrounding an outer surface of the nozzle and the ground electrode includes a second metal ring surrounding an outer surface of the nozzle.

4. The plasma brush of claim 3, wherein the first metal ring and the second metal ring have the same vertical height.

5. The plasma brush of claim 3, wherein a lateral width of the first metal ring is less than that of the second metal ring.

6. The plasma brush of claim 3, wherein the nozzle includes a first groove in which the first metal ring is disposed and a second groove in which the second metal ring is disposed.

7. The plasma brush of claim 6, wherein the nozzle further includes a protruding sill that is disposed between the first and second grooves to separate the first metal ring from the second metal ring.

8. The plasma brush of claim 2, further comprising a filler nozzle inserted into the discharge port,
wherein the filler nozzle has a plurality of pores for ejecting a gas.

9. The plasma brush of claim 8, wherein the ground electrode fills a space between the pores to have a grid shape or a mesh shape.

10. The plasma brush of claim 9, wherein the ground electrode is disposed on a front surface of the filler nozzle, and wherein the plasma brush further comprises a first coating layer covering a front surface of the ground electrode opposite to filler nozzle.

11. The plasma brush of claim 2, wherein the power electrode includes a plurality of needles disposed in the discharge port.

12. The plasma brush of claim 11, further comprising a conductive adhesive agent that electrically connects the power needles to each other.

13. The plasma brush of claim 11, further comprising a second coating layer that protects the power needles.

14. The plasma brush of claim 1, wherein the nozzle includes a dielectric material.

15. The plasma brush of claim 14, wherein the dielectric material includes an alumina material or a ceramic material.

16. The plasma brush of claim 1, wherein the connector includes a tube having a plurality of channel holes through which a gas flows.

17. The plasma brush of claim 16, wherein the tube includes an insulation rod surrounded by the plurality of channel holes.

18. The plasma brush of claim 16, wherein the connector further includes a third coating layer protecting the tube.
